(19) United States
(12) Patent Application Publication
(10) Pub. No.: US 2018/0342173 A1
(43) Pub. Date: Nov. 29, 2018

(54) Method of Monitoring Glucose Levels for Weight Loss

(71) Applicant: Winston Jordan, Lambertville, NJ (US)

(72) Inventor: Winston Jordan, Lambertville, NJ (US)

(21) Appl. No.: 15/988,609

(22) Filed: May 24, 2018

Related U.S. Application Data
(60) Provisional application No. 62/510,640, filed on May 24, 2017.

(51) Int. Cl. G09B 19/00; A61B 5/00; G16H 20/60; G16H 40/63; G01N 33/49; G01N 33/487

(52) U.S. Cl. CPC G09B 19/0092; A61B 5/486; A61B 5/4872; G01N 33/48792; G16H 40/63; G01N 33/4925; G16H 20/60

(57) ABSTRACT

The method of monitoring glucose levels begins with the retrieval of at least one daily glucose reading with a user personal computing (PC) device. A daily weight measurement is also retrieved with the user PC device. A daily biometric summary is then relayed from the user PC device to a remote server, wherein the daily biometric summary includes the daily glucose reading and the daily weight measurement. A plurality of iterations is executed in order to compile a user biometric profile, wherein the user biometric profile includes the daily biometric summary for each iteration. The user biometric profile is then compared to the ideal biometric profile with the remote server in order to generate a biometric assessment for the user account. The biometric assessment is outputted with the user PC device for the viewing of the user account.

The current application claims priority to U.S. provisional application Ser. No. 62/510,640 filed on May 24, 2017.

### FIELD OF THE INVENTION

The present invention generally relates to weight loss management and overall well-being improvement. More specifically, the present invention allows a user to keep track of his or her body weight, glucose levels, and body fat percentage.

### BACKGROUND OF THE INVENTION

The methodology for weight loss in recent decades has primarily focused on strictly food and exercise. However, recent developments have led to the understanding that weight loss is also dependent on the glucose concentration and the rate of processing, more specifically, the secretion of glucagon in the bloodstream. Diabetics have analyzed glucose concentrations in order to mitigate dangerous glucose levels. This methodology has not been utilized to solely cater to weight loss.

It is therefore the objective of this invention to provide an application that uses such a methodology which revolves around glucose readings. The present invention more specifically concentrates on the energy expenditure with respect to glucose levels. Moreover, the present invention serves to effectively aid in weight-loss in comparison to traditional monitoring of caloric energy. The present invention warns the user of hyperglycemia. As a user is typically unhealthy, glucose levels must be monitored before a meal in order to determine if the glucose level of the user is in an optimal range and the meal will not increase an existing or near hypoglycemic reading. The present invention tracks the compliance and success of a user. The present invention supplies the user with an analysis of his or her progress and provides supplemental information that is unique to the progress and overall well-being of the user.

### BRIEF DESCRIPTION OF THE DRAWINGS

FIG. 1 is a schematic view for a physical system of the present invention.

FIG. 2 is a flowchart illustrating the overall process of the present invention.

FIG. 3 is a flowchart illustrating the process for retrieving a pre-meal glucose reading through a user personal computing (PC) device of the present invention.

FIG. 4 is a flowchart illustrating the process for retrieving a post-meal glucose reading through the user PC device of the present invention.

FIG. 5 is a flowchart illustrating the process for appending a meal-content summary for the meal glucose reading into a daily biometric summary with the user PC device of the present invention.

FIG. 6 is a flowchart illustrating the process for appending a meal picture for a meal glucose reading into the daily biometric summary with the user PC device of the present invention.

FIG. 7 is a flowchart illustrating the process for appending a daily body-fat measurement for a daily glucose reading into the daily biometric summary with the user PC device of the present invention.

FIG. 8 is a flowchart illustrating the process for relaying the daily glucose reading from a glucometer to the user PC device of the present invention.

FIG. 9 is a flowchart illustrating the process for receiving a user input as the daily glucose reading through the user PC device of the present invention.

FIG. 10 is a flowchart illustrating the process for immediately outputting either a goal-reached notification or a goal-unreached notification with the user PC device after a latest iteration of the present invention.

FIG. 11 is a flowchart illustrating the process for appending an additional glucose reading into the user biometric profile with the remote server of the present invention.

FIG. 12 is a flowchart illustrating the process for immediately outputting either the goal-reached notification or the goal-unreached notification with the user PC device after a plurality of sequential iterations of the present invention.

FIG. 13 is a flowchart illustrating the process for outputting a nutritionally-matching suggestion with the user PC device of the present invention.

FIG. 14 is a flowchart illustrating the process for outputting a contextually-matching article with the user PC device of the present invention.

FIG. 15 is a flowchart illustrating the process for outputting a summarization report with the user PC device of the present invention.

FIG. 16 is a flowchart illustrating the process for outputting a goal-reached notification with the user PC device, if a current biometric measurement matches a desired biometric characteristic of the present invention.

FIG. 17 is a flowchart illustrating the process for posting the meal picture and the goal-reached notification on the user profile with the remote server of the present invention.

FIG. 18 is a flowchart illustrating the process for outputting a projective portion for an advanced biometric history of a biometrically-similar account with the user PC device of the present invention.

FIG. 19 is a schematic view a dashboard of the present invention as seen on a screen of the user PC device.

### DETAIL DESCRIPTIONS OF THE INVENTION

All illustrations of the drawings are for the purpose of describing selected versions of the present invention and are not intended to limit the scope of the present invention.

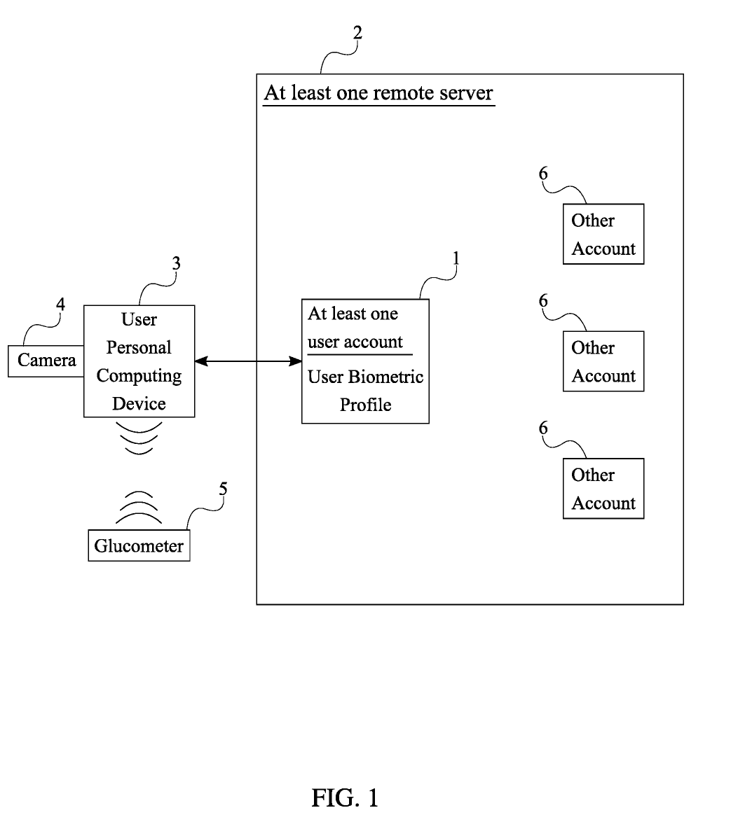

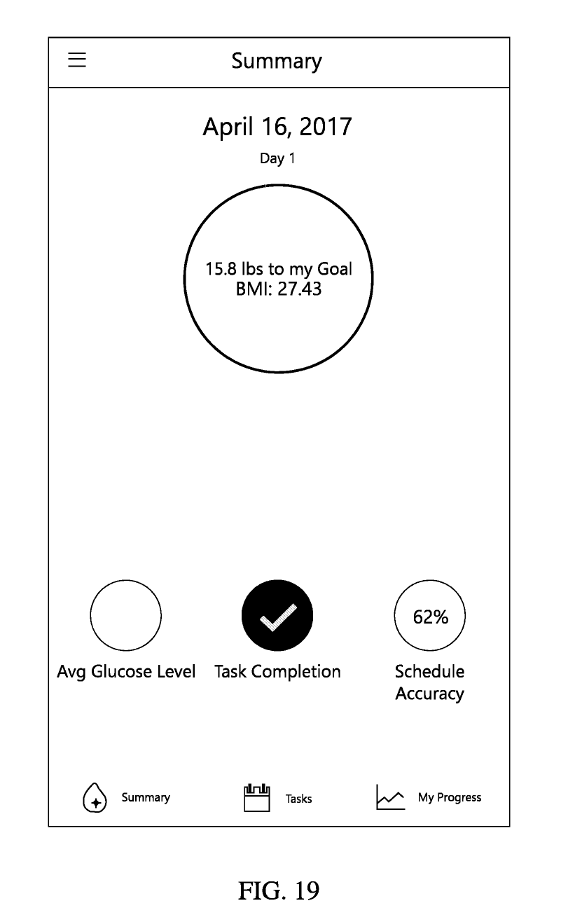

The present invention is a method of monitoring glucose levels. More specifically, the present invention allows a user to monitor and record a variety of biometric readings in order to achieve a desired weight and overall well-being. The physical system used to implement the method of the present invention includes at least one remote server 2 and a user personal computing user (PC) device 3, as seen in FIG. 1. The at least one remote server 2 manages at least one user account 1. The user account 1 is associated to the user PC device 3 and to an ideal biometric profile (Step A). The user PC device 3 communicates with the server and delivers biometric readings associated with the user account 1 to the server. The ideal biometric profile serves as a benchmark or standard that allows the biometric readings to be properly accessed. The user PC device 3 is preferably a smart phone that utilizes a camera 4, a display screen, and a processing unit of the smartphone. A dashboard of the present invention is illustrated in FIG. 19 as viewed by the user account 1 with the user PC device 3. Alternate embodiments of the present invention may have a user PC device 3 that is computer, smart watch, tablet, and so on. The user account 1 is able to register and enter vitals with the user PC device 3 in order to differentiate the user account 1 among other user accounts. More specifically, the user account 1 registers with the present invention by designating a password, a username, and a variety of other identification and verification information.

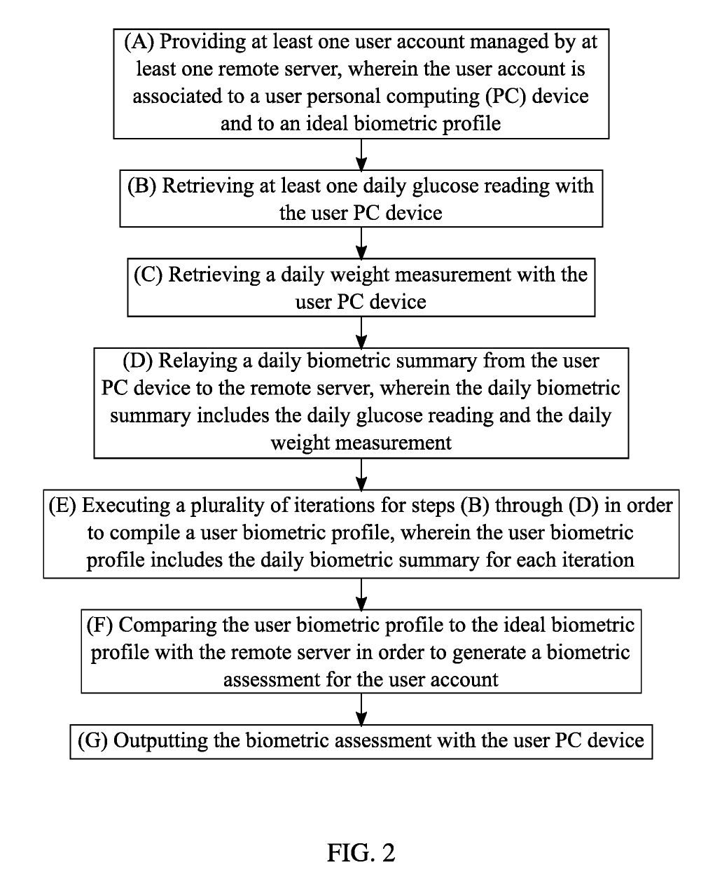

The overall process for the present invention includes the following steps that are implemented with at least one remote server 2 and the user PC device 3. As shown in FIG. 2, the overall process begins by retrieving at least one daily glucose reading with the user PC device 3 (Step B). The at least one daily glucose reading provides more accurate information regarding the health of the user account 1. In order to provide a more in-depth analysis of the current state of health of the user account 1 as well as the progress of the user account 1, a daily weight measurement is retrieved with the user PC device 3 (Step C). A daily biometric summary is relayed from the user PC device 3 to the remote server 2, wherein the daily biometric summary includes the daily glucose reading and daily weight measurement (Step D), thereby recording all input by the user account 1. A plurality of iterations is executed for steps (B) through (D) in order to compile a user biometric profile, wherein the user biometric profile includes the daily biometric summary for each iteration (Step E). Moreover, the plurality of iterations account for each meal of the user account 1 and glucose readings of the at least one daily glucose reading associated with each meal. The user biometric profile is then compared to the ideal biometric profile with the remote server 2 in order to generate a biometric assessment for the user account 1 (Step F). The biometric assessment informs the user account 1 of the current state of health based. In order for the user account 1 to review the biometric assessment, the biometric assessment is outputted with the user PC device 3 (Step G).

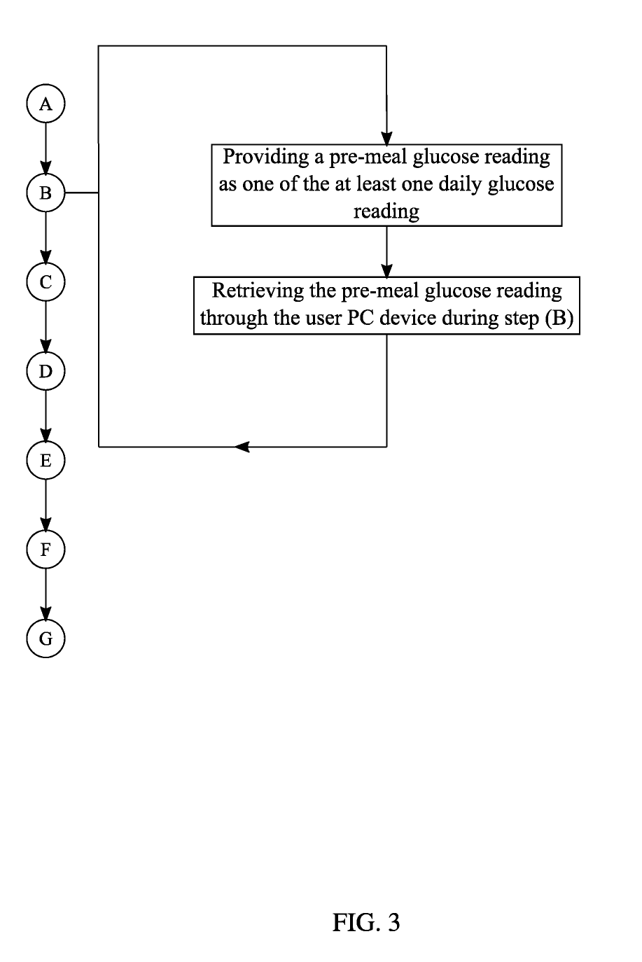

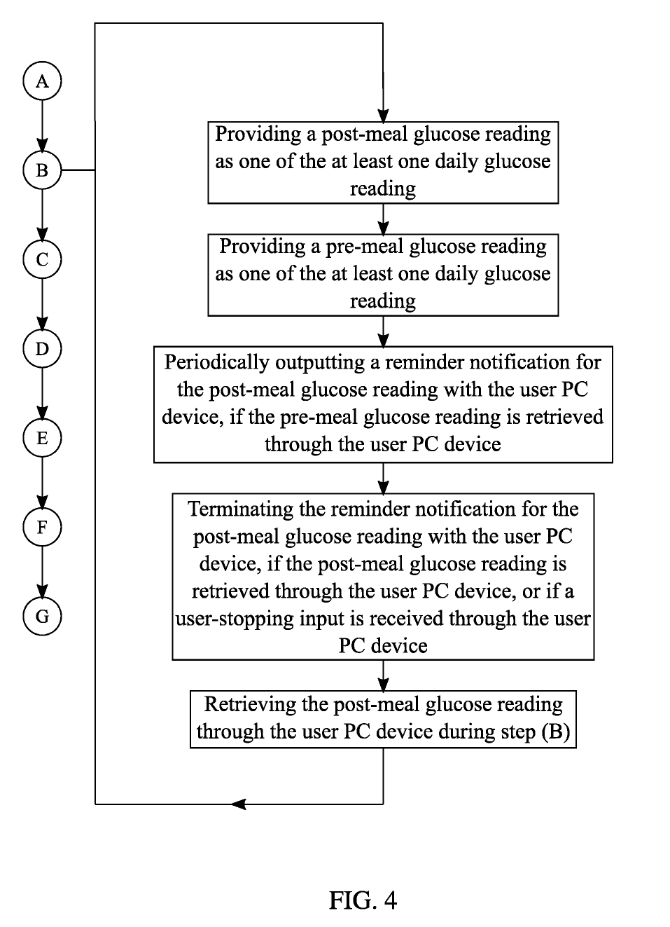

The present invention provides a more accurate biometric assessment as a pre-meal glucose reading is provided as one of the at least one daily glucose reading. The pre-meal glucose reading is a glucose reading that is taken before the user account 1 has a meal. As seen in FIG. 3, the pre-meal glucose reading is retrieved through the user PC device 3 during step (B). Similarly, a post-meal glucose reading is provided as one of the at least one daily glucose reading. As seen in FIG. 4, the post-meal glucose reading is retrieved through the user PC device 3 during step (B). The pre-meal glucose reading and the post-meal glucose reading allows the at least one remote server 2 to determine the impact of the meal on the glucose level of the user account 1. Moreover, the pre-meal glucose reading and the post-meal glucose reading provide the biometric assessment with more data in order to provide a more accurate assessment of the health of the user.

In the preferred embodiment of the present invention, a reminder notification is periodically outputted for the post-meal glucose reading with the user PC device 3, if the pre-meal glucose reading is retrieved through the user PC device 3, also seen in FIG. 4. Moreover, the pre-meal glucose reading initiates a meal cycle that reminds the user account 1 that the post-meal glucose reading should be inputted in order to provide a more accurate biometric assessment. The reminder notification for the post-meal glucose reading with the user PC device 3 is terminated, if the post-meal glucose reading is retrieved through the user PC device 3. In the event the post-meal glucose reading is not able to be inputted, the reminder notification for the post-meal glucose reading is terminated with the user PC device 3 if a user-stopping input is received though the user PC device 3.

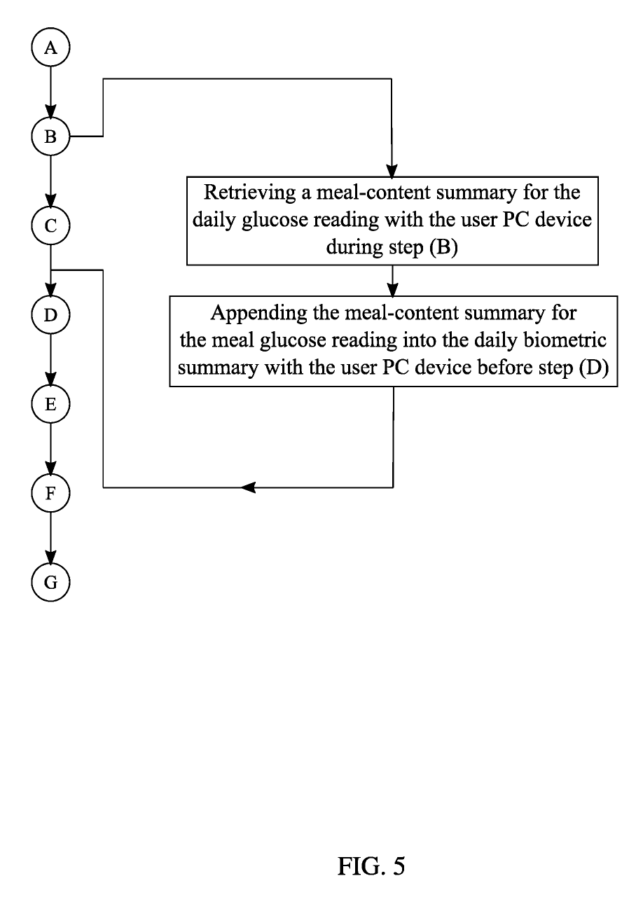

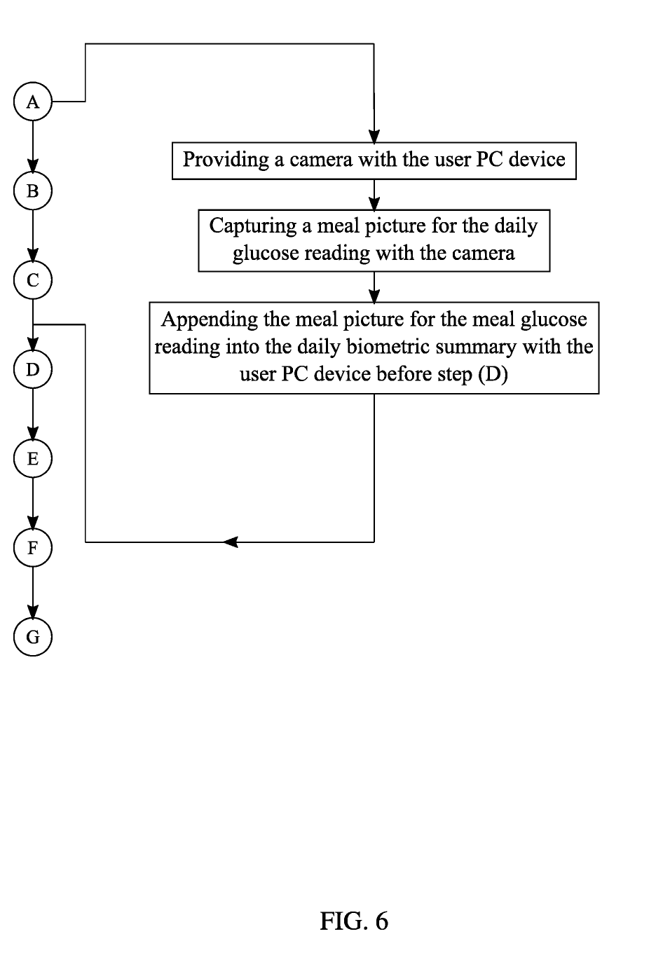

As a meal affects the glucose level of the user, which may be accounted for with the post-meal glucose reading, a meal-content summary for the daily glucose reading is retrieved with the user PC device 3 during step (B), seen in FIG. 5. This allows the biometric assessment to provide an accurate analysis as to why the health of the user account 1 is improving or declining. The meal-content summary is then appended for the meal glucose reading into the daily biometric summary with the user PC device 3 before step (D). Furthermore, in the preferred embodiment of the present invention, a camera 4 with the user PC device 3 is provided in order to associate an image of a meal with the corresponding meal-content summary, pre-glucose reading, and post-glucose reading. As seen in FIG. 6, a meal picture is captured for the daily glucose reading with the camera 4. The meal picture is then appended into the daily biometric summary with the user PC device 3 before step (D). The meal picture may motivate the user account 1 to eat healthier and cleaner meals as well as other user accounts 1.

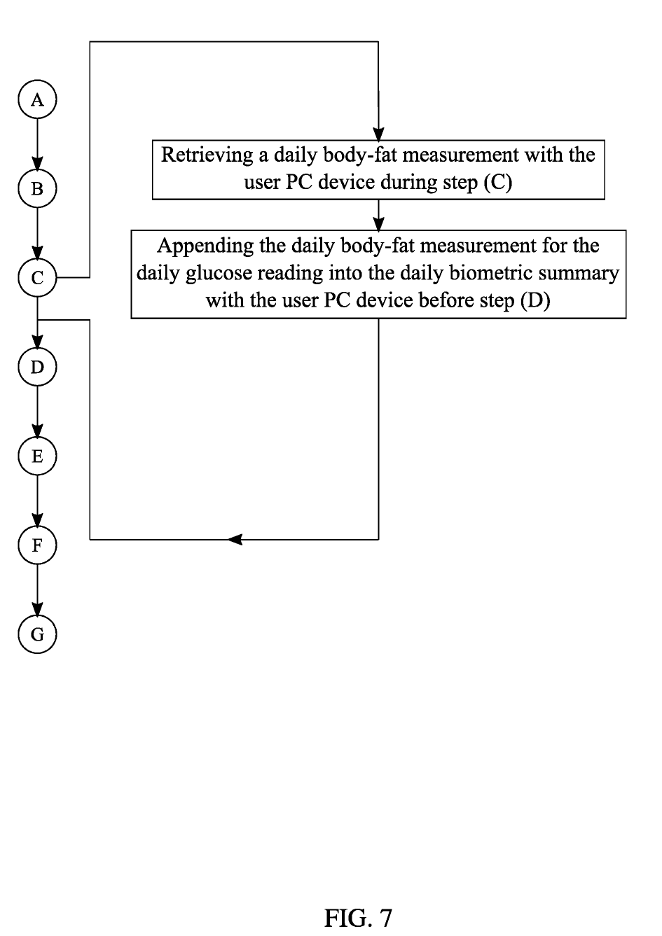

In the preferred embodiment of the present invention, a daily body-fat measurement is retrieved with the user PC device 3 during step (C), seen in FIG. 7. The daily body-fat measurement provides a more accurate biometric assessment in addition to the daily weight measurement and the at least one daily glucose reading. The daily body-fat measurement may be manually taken or automatically taken with handheld body fat monitor. The daily body-fat measurement then is appended for the daily glucose reading into the daily biometric summary with the user PC device 3 before step (D).

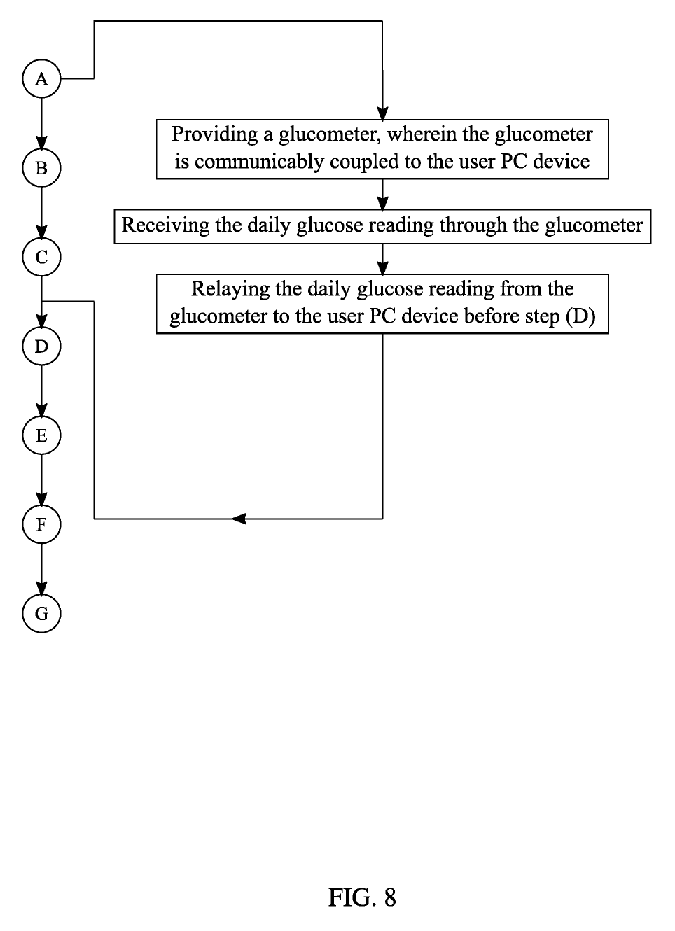

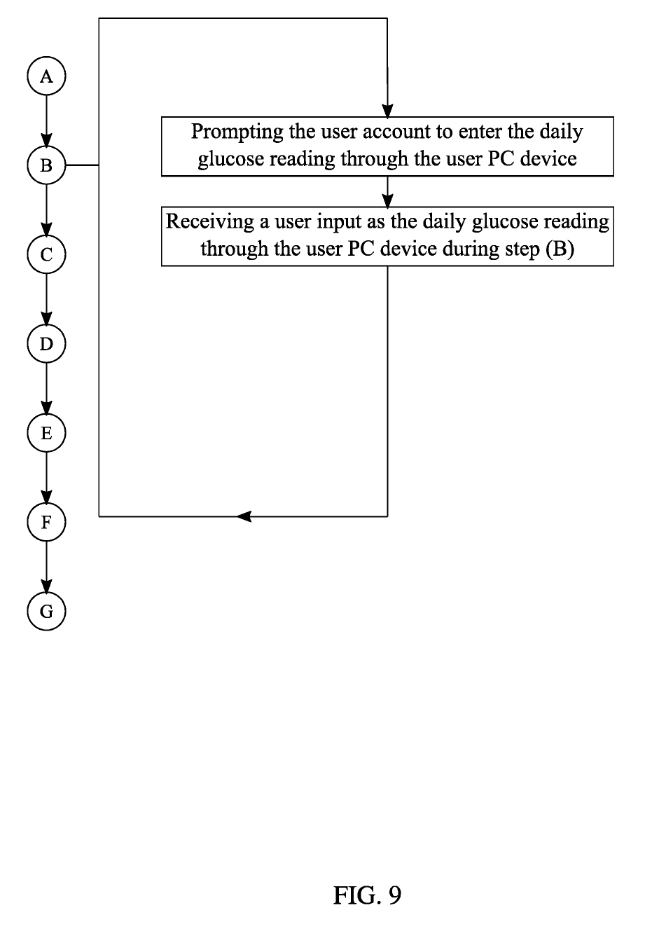

In another embodiment of the present invention, a glucometer 5 is provided, wherein the glucometer 5 is communicably coupled to the user PC device 3. The glucometer 5 automatically delivers a daily glucose reading to the user PC device 3 so that that user account 1 does not have to manually input the daily glucose reading into the user PC device 3. Moreover, the daily glucose reading is received through the glucometer 5, seen in FIG. 8. The daily glucose reading is then relayed from the glucometer 5 to the user PC device 3 before step (D). However, the glucose reading may be taken in a variety of ways with a variety of tools that may not be communicably coupled to the user PC device 3. Therefore, in the preferred embodiment of the present invention, the user account 1 is prompted to enter the daily glucose reading through the user PC device 3, as seen in FIG. 9. A user input is then received as the daily glucose reading through the user PC device 3 during step (B).

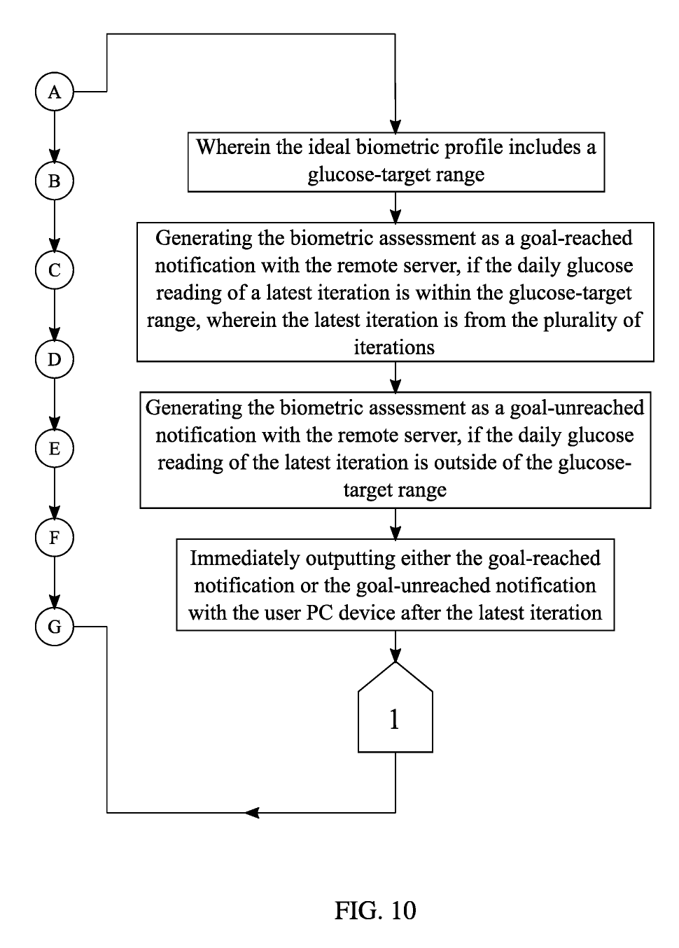

In order for the user account 1 to determine whether or not the at least one daily glucose reading indicates that the user is making progress reaching a goal, the ideal biometric profile includes a glucose-target range, as shown in FIG. 10. The glucose-target range defines an average range of glucose readings. The biometric assessment generates a goal-reached notification with the remote server 2, if the daily glucose reading of a latest iteration is within the glucose-target range, wherein the latest iteration is from the plurality of iterations. In the preferred embodiment of the present invention, the goal-reached notification is associated with a color, preferably green. The color association of the goal-reached notification visually enhances the presentation of the goal-reached notification with the user PC device 3. Similarly, the biometric assessment generates a goal-unreached notification with the remote server 2, if the daily glucose reading of the latest iteration is outside of the glucose target range. In the preferred embodiment of the present invention, the goal-unreached notification is associated with another color, preferably red. Moreover, if the biometric assessment generates a goal-reached notification, where the daily glucose reading is average, the goal-reached notification may be associated with another color, preferably yellow. The user account 1 is able to view the goal-reached notification and the goal-unreached notification as either the goal-reached notification or the goal-unreached notification is immediately outputted with the user PC device 3 after the latest iteration.

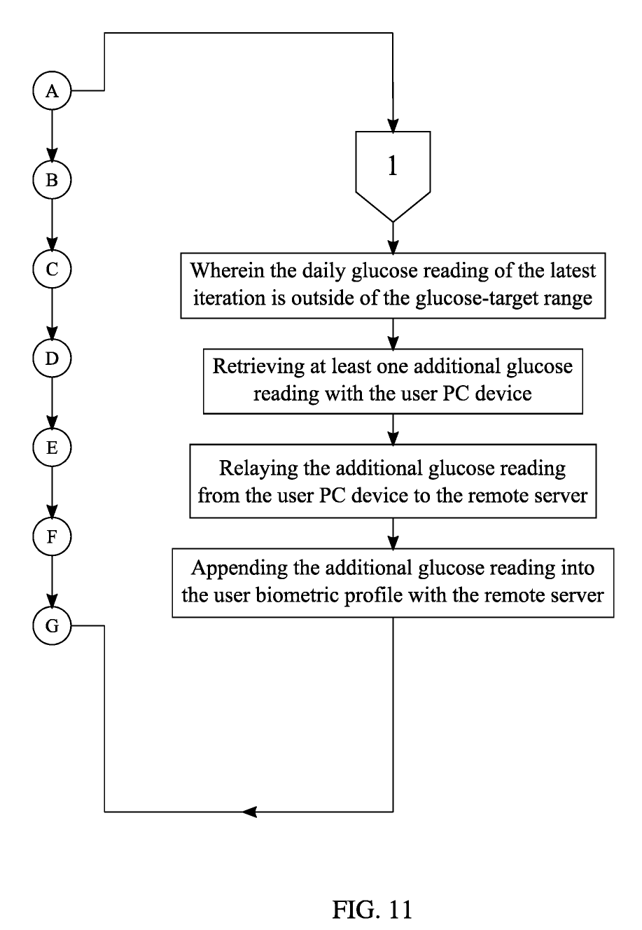

The present invention ensures the well-being of the user account 1 as at least one additional glucose reading is retrieved with the user PC device 3 when the daily glucose reading of the latest iteration is outside of the glucose target range, seen in FIG. 11. The additional glucose reading is relayed from the user PC device 3 to the remote server 2 and is then appended into the user biometric profile with remote server 2. In addition to ensuring the well-being of the user account 1, the additional glucose reading serves as additional data for the user biometric profile, thereby increasing the accuracy of the user biometric profile.

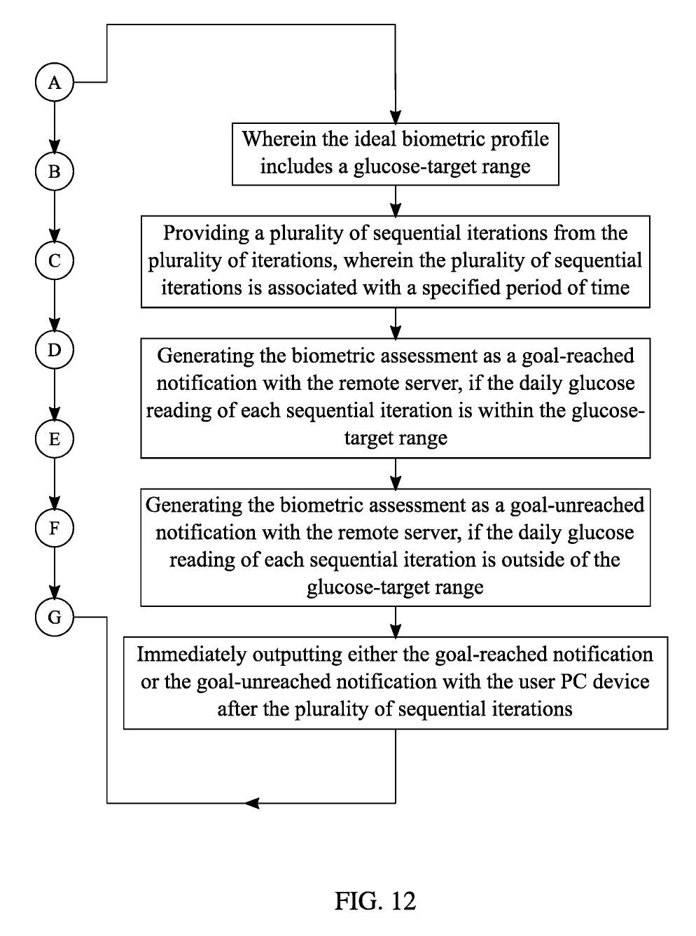

The present invention provides further analysis for the progress of the user as a plurality of sequential iterations from the plurality of iterations is provided, wherein the plurality of sequential iterations is associated with a specific period of time, as seen in FIG. 12. Moreover, the plurality of sequential iterations over a specific period of time allows the user account 1 to analyze progress over a week, a month, a year, and so on. The biometric assessment is generated as a goal-oriented notification with the remote server 2, if the daily glucose reading of each sequential iteration is within the glucose-target range. Similarly, the biometric assessment is generated as a goal-unreached notification with the remote server 2, if the daily glucose reading of each sequential iteration is outside of the glucose target range. The user account 1 is able to review progress over the specific period of time as the goal-reached notification or the goal-unreached notification is immediately outputted with the user PC device 3 after the plurality of sequential iterations.

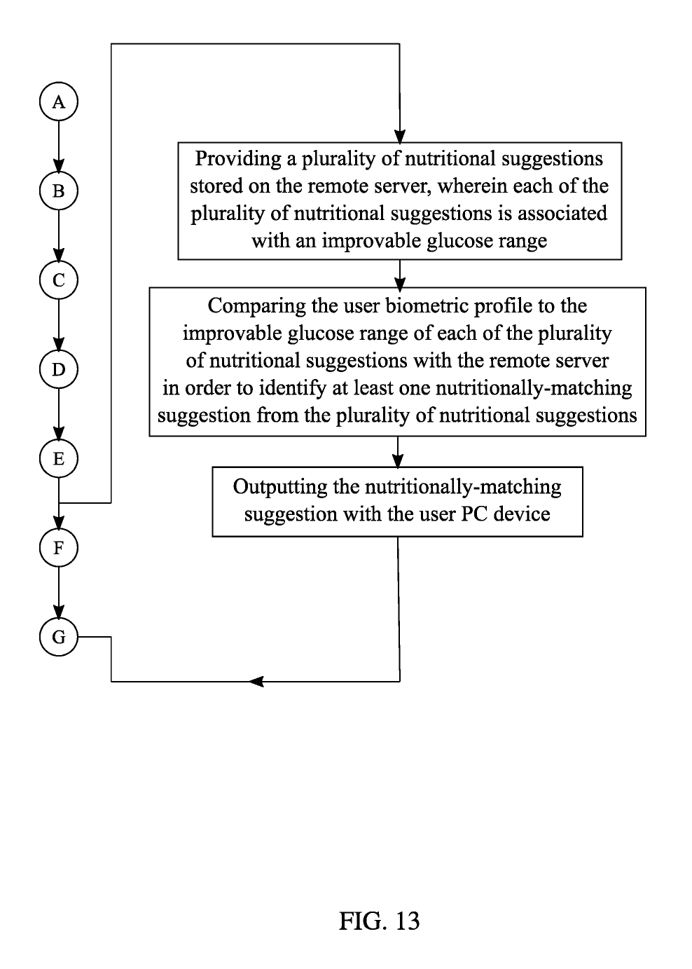

As seen in FIG. 13, the preferred embodiment of the present invention further provides a plurality of nutritional suggestions stored on the remote server 2. Each of the plurality of nutritional suggestions is associated with an improvable glucose range. Moreover, the plurality of nutritional suggestions serves as a set of recommendations for specific compounds. Such compounds may include, but are not limited to, vitamins, minerals, and certain amino acids. In addition to specific compounds, the plurality of nutritional suggestions may include a portion size as well. The plurality of nutritional suggestions is based on the user biometric profile of the user account 1. The user biometric profile is compared to the improvable glucose range of each of the plurality of nutritional suggestions with the remote server 2 in order to identify at least one nutritionally-matching suggestion from the plurality of nutritional suggestions, thereby accounting for the current state of health of the user account 1 and the desired health goal of the user account 1. The nutritionally-matching suggestion is then outputted with the user PC device 3 so that the user account 1 may view the nutritionally-matching suggestion.

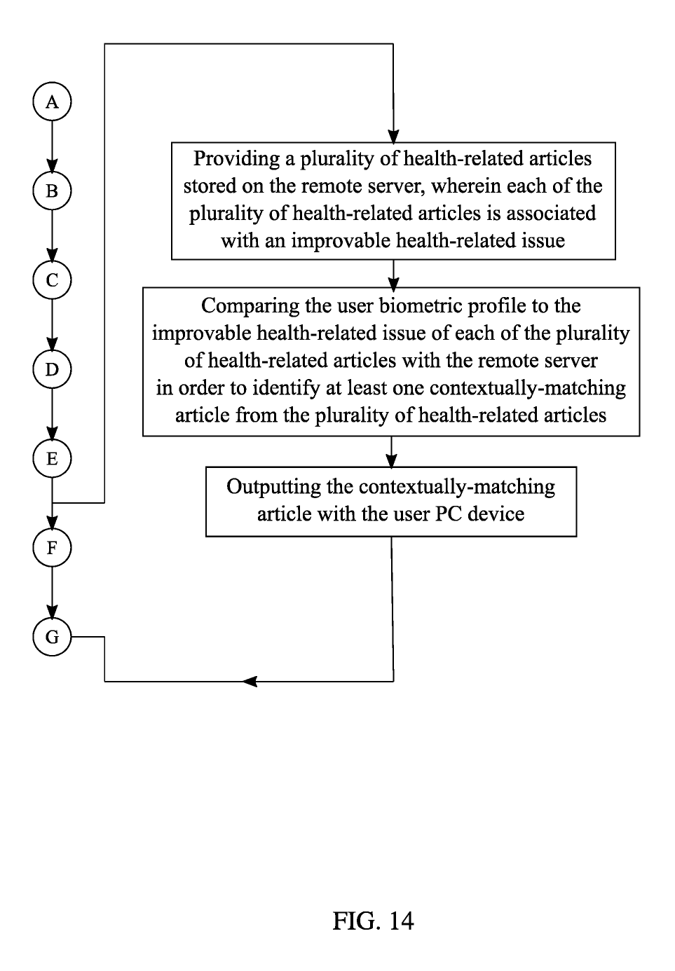

As seen in FIG. 14, the preferred embodiment of the present invention further provides a plurality of health-related articles stored on the remote server 2. Similar to the plurality of nutritional suggestions, the plurality of health-related articles provide helpful information that may aid in the progress of the user account 1. Moreover, each of the plurality of health-related articles is associated with an improvable health-related issue. The user biometric profile is compared to the improvable health-related issue of each of the plurality of health-related articles with the remote server 2 in order to identify at least one contextually-matching article from the plurality of health-related articles, thereby providing the user account 1 with accurate and relevant health-related articles. The contextually-matching article is outputted with the user PC device 3 in order for the user account 1 to be able to view the contextually-matching article.

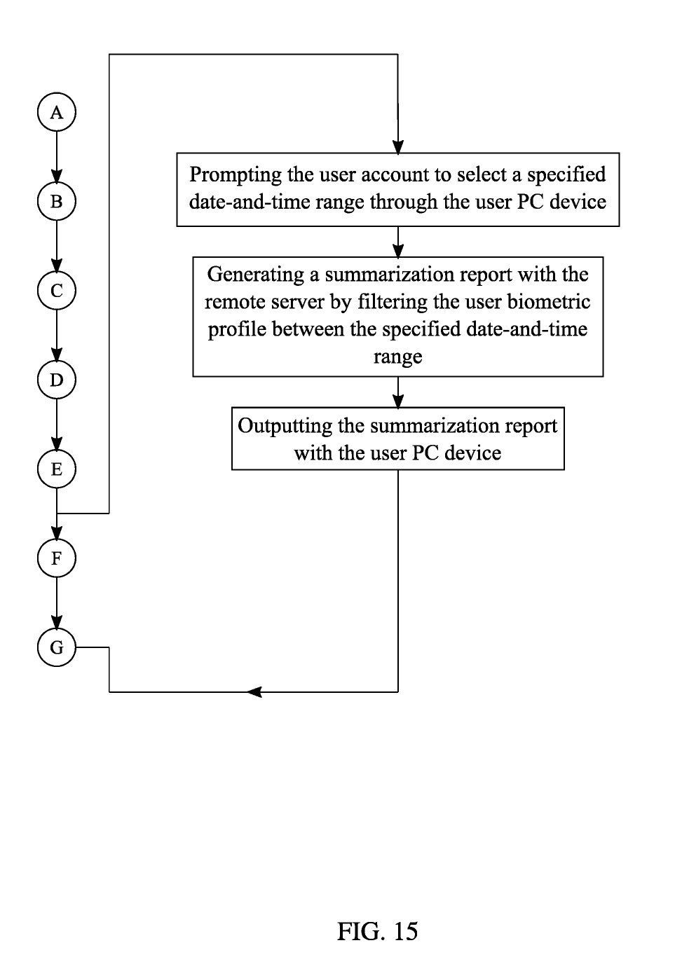

In order to provide the user account 1 with visual aids which present the progress of the user account 1, the user account 1 is prompted to select a specified date-and-time range through the user PC device 3, as seen in FIG. 15. The specified date-and-time range defines the time as a variable in the analysis for the progress of the user account 1. A summarization report is generated with the remote server 2 by filtering the user biometric profile between the specified date-and-time range. The summarization report is then outputted with the user PC device 3. The summarization report may include, but is not limited to, bar graphs, pie charts, line charts.

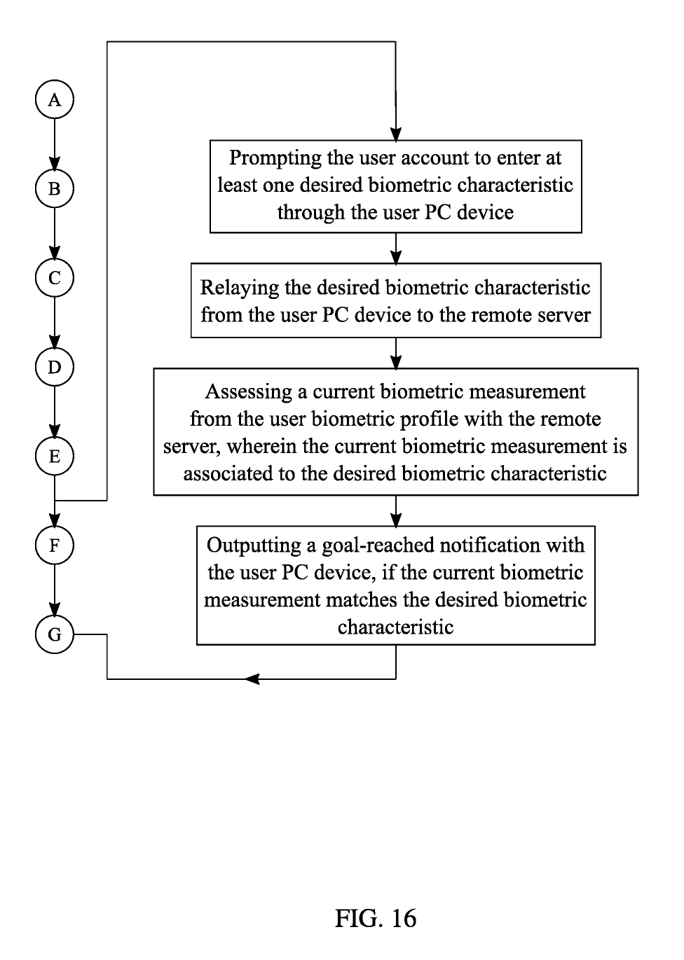

Throughout the daily inputs for weight and at least one glucose readings, the present invention alerts the user account 1 if the goal of the user account 1 has been reached. In order for the present invention to determine if the goal of the user account 1 is reached, the user account 1 is prompted to enter at least one desired biometric characteristic through the user PC device 3, as seen in FIG. 16. The at least one desired biometric characteristic may be specific readings, desired physical features that may be determined by requested inputs of the user account 1, and so on. The desired biometric characteristic is relayed from the user PC device 3 to the remote server 2. A current biometric measurement from the user biometric profile is assessed with the remote server 2, wherein the current biometric measurement is associated to the desired biometric characteristic. The user account 1 is alerted of the accomplishment as a goal-reached notification is outputted with the user PC device 3, if the current biometric measurement matches the desired biometric characteristic.

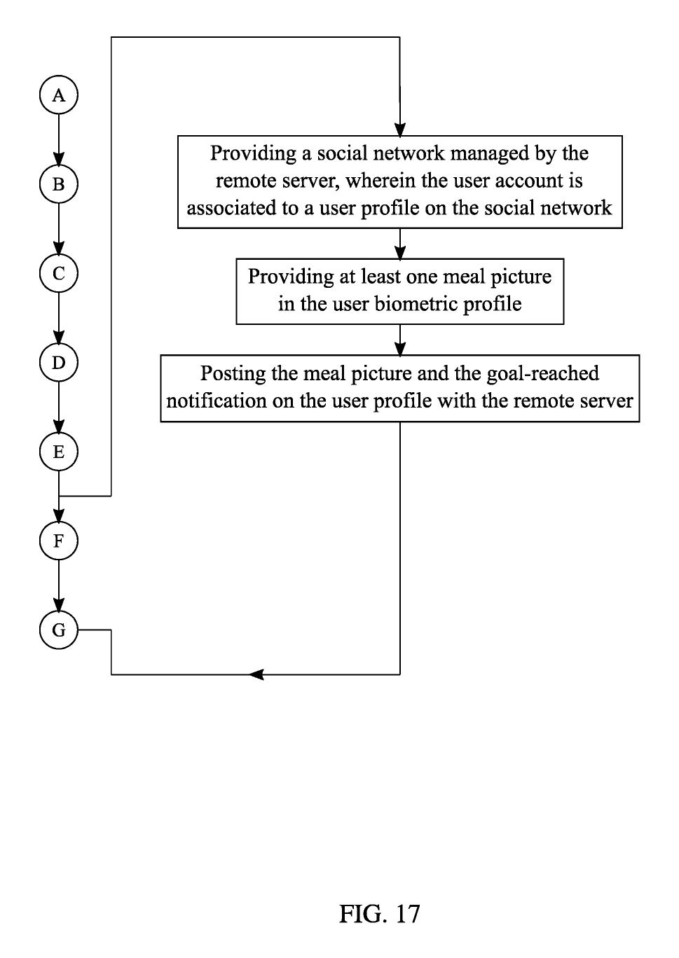

The present invention also acknowledges the progress of the user accounts and aids other user accounts with similar user biometric profiles. In order to acknowledge and aid other user accounts, a social network is provided by the remote server 2, wherein the user account 1 is associated to a user profile on the social network, seen in FIG. 17. Furthermore, at least one meal picture is provided in the user biometric profile. The at least one meal picture is an image of healthy foods consumed by the user account 1. The meal picture and the goal-reached notification is posted on the user profile with the remote server 2 allowing other user accounts with similar user biometric profiles to view the meal picture. The meal picture may inspire the other user accounts and aid the progress of the user account 1.

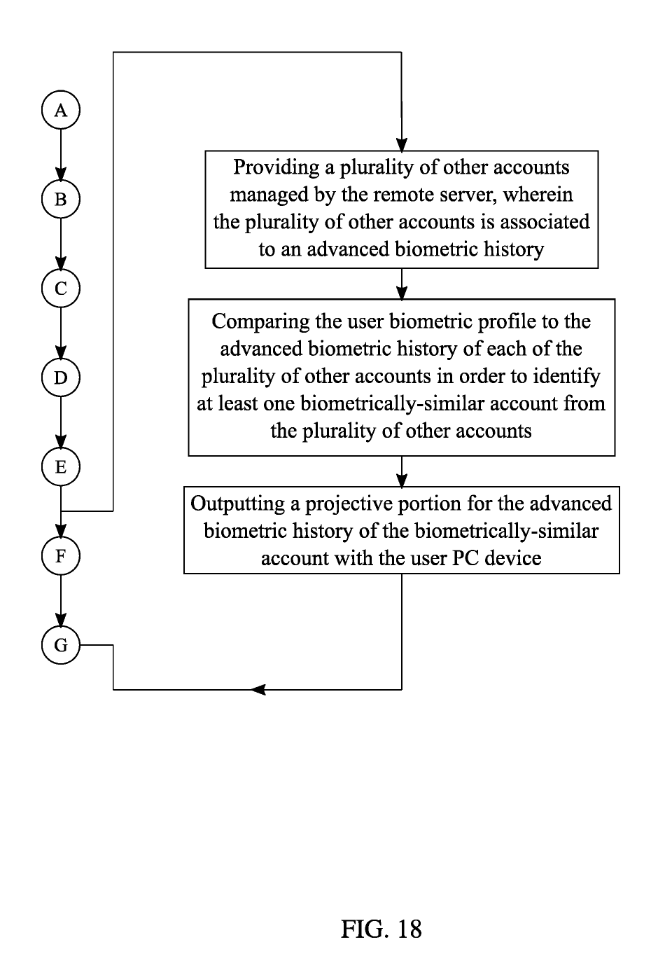

The present invention further aids in the progress of the user account 1 by inspiring the user account 1 with other user accounts. More specifically, as seen in FIG. 18, a plurality of other accounts 6 managed by the remote server 2 is provided. The plurality of other accounts 6 is associated to an advanced biometric history. Each advanced biometric history includes progress of a plurality of accounts of which is expected of the progress of the user account 1. The user biometric profile is compared to the advanced biometric history of each of the plurality of other accounts 6 in order to identify at least on biometrically-similar account from the plurality of other accounts 6. A projective portion is then outputted for the advanced biometric history of the biometrically-similar account with the user PC device 3. The progress of the user account 1 may be visually compared to the at least one biometrically-similar account, hopefully aiding the user with health-related choices. The projective portion allows the user account 1 to better track progress with the present invention.

Although the invention has been explained in relation to its preferred embodiment, it is to be understood that many other possible modifications and variations can be made without departing from the spirit and scope of the invention as hereinafter claimed.

What is claimed is:

1. A method of monitoring glucose levels for weight loss, the method comprises the steps of:
(A)providing at least one user account managed by at least one remote server, wherein the user account is associated to a user personal computing (PC) device and to an ideal biometric profile;
(B) retrieving at least one daily glucose reading with the user PC device;
(C) retrieving a daily weight measurement with the user PC device;
(D)relaying a daily biometric summary from the user PC device to the remote server, wherein the daily biometric summary includes the daily glucose reading and the daily weight measurement;
(E) executing a plurality of iterations for steps (B) through (D) in order to compile a user biometric profile, wherein the user biometric profile includes the daily biometric summary for each iteration;
(F) comparing the user biometric profile to the ideal biometric profile with the remote server in order to generate a biometric assessment for the user account; and
(G) outputting the biometric assessment with the user PC device.

2. The method of monitoring glucose levels for weight loss, the method as claimed in claim 1 comprises the steps of:
providing a pre-meal glucose reading as one of the at least one daily glucose reading; and
retrieving the pre-meal glucose reading through the user PC device during step (B).

3. The method of monitoring glucose levels for weight loss, the method as claimed in claim 1 comprises the steps of:
providing a post-meal glucose reading as one of the at least one daily glucose reading; and
retrieving the post-meal glucose reading through the user PC device during step (B).

4. The method of monitoring glucose levels for weight loss, the method as claimed in claim 3 comprises the steps of:
providing a pre-meal glucose reading as one of the at least one daily glucose reading;
periodically outputting a reminder notification for the post-meal glucose reading with the user PC device, if the pre-meal glucose reading is retrieved through the user PC device; and
terminating the reminder notification for the post-meal glucose reading with the user PC device, if the post-meal glucose reading is retrieved through the user PC device, or if a user-stopping input is received through the user PC device.

5. The method of monitoring glucose levels for weight loss, the method as claimed in claim 1 comprises the steps of:
retrieving a meal-content summary for the daily glucose reading with the user PC device during step (B); and
appending the meal-content summary for the meal glucose reading into the daily biometric summary with the user PC device before step (D).

6. The method of monitoring glucose levels for weight loss, the method as claimed in claim 1 comprises the steps of:
providing a camera with the user PC device;
capturing a meal picture for the daily glucose reading with the camera; and
appending the meal picture for the meal glucose reading into the daily biometric summary with the user PC device before step (D).

7. The method of monitoring glucose levels for weight loss, the method as claimed in claim 1 comprises the steps of:
retrieving a daily body-fat measurement with the user PC device during step (C); and
appending the daily body-fat measurement for the daily glucose reading into the daily biometric summary with the user PC device before step (D).

8. The method of monitoring glucose levels for weight loss, the method as claimed in claim 1 comprises the steps of:
providing a glucometer, wherein the glucometer is communicably coupled to the user PC device;
receiving the daily glucose reading through the glucometer; and
relaying the daily glucose reading from the glucometer to the user PC device before step (D).

9. The method of monitoring glucose levels for weight loss, the method as claimed in claim 1 comprises the steps of:
prompting the user account to enter the daily glucose reading through the user PC device; and
receiving a user input as the daily glucose reading through the user PC device during step (B).

10. The method of monitoring glucose levels for weight loss, the method as claimed in claim 1 comprises the steps of:
wherein the ideal biometric profile includes a glucose-target range;
generating the biometric assessment as a goal-reached notification with the remote server, if the daily glucose reading of a latest iteration is within the glucose-target range, wherein the latest iteration is from the plurality of iterations;
generating the biometric assessment as a goal-unreached notification with the remote server, if the daily glucose reading of the latest iteration is outside of the glucose-target range; and
immediately outputting either the goal-reached notification or the goal-unreached notification with the user PC device after the latest iteration.

11. The method of monitoring glucose levels for weight loss, the method as claimed in claim 10 comprises the steps of:
wherein the daily glucose reading of the latest iteration is outside of the glucose-target range;
retrieving at least one additional glucose reading with the user PC device;
relaying the additional glucose reading from the user PC device to the remote server; and
appending the additional glucose reading into the user biometric profile with the remote server.

12. The method of monitoring glucose levels for weight loss, the method as claimed in claim 1 comprises the steps of:
wherein the ideal biometric profile includes a glucose-target range;
providing a plurality of sequential iterations from the plurality of iterations, wherein the plurality of sequential iterations is associated with a specified period of time;
generating the biometric assessment as a goal-reached notification with the remote server, if the daily glucose reading of each sequential iteration is within the glucose-target range;
generating the biometric assessment as a goal-unreached notification with the remote server, if the daily glucose reading of each sequential iteration is outside of the glucose-target range; and
immediately outputting either the goal-reached notification or the goal-unreached notification with the user PC device after the plurality of sequential iterations.

13. The method of monitoring glucose levels for weight loss, the method as claimed in claim 1 comprises the steps of:
providing a plurality of nutritional suggestions stored on the remote server, wherein each of the plurality of nutritional suggestions is associated with an improvable glucose range;
comparing the user biometric profile to the improvable glucose range of each of the plurality of nutritional suggestions with the remote server in order to identify at least one nutritionally-matching suggestion from the plurality of nutritional suggestions; and
outputting the nutritionally-matching suggestion with the user PC device.

14. The method of monitoring glucose levels for weight loss, the method as claimed in claim 1 comprises the steps of:
providing a plurality of health-related articles stored on the remote server, wherein each of the plurality of health-related articles is associated with an improvable health-related issue;
comparing the user biometric profile to the improvable health-related issue of each of the plurality of health-related articles with the remote server in order to identify at least one contextually-matching article from the plurality of health-related articles; and
outputting the contextually-matching article with the user PC device.

15. The method of monitoring glucose levels for weight loss, the method as claimed in claim 1 comprises the steps of:
prompting the user account to select a specified date-and-time range through the user PC device;
generating a summarization report with the remote server by filtering the user biometric profile between the specified date-and-time range; and
outputting the summarization report with the user PC device.

16. The method of monitoring glucose levels for weight loss, the method as claimed in claim 1 comprises the steps of:
prompting the user account to enter at least one desired biometric characteristic through the user PC device;
relaying the desired biometric characteristic from the user PC device to the remote server;
assessing a current biometric measurement from the user biometric profile with the remote server, wherein the current biometric measurement is associated to the desired biometric characteristic; and
outputting a goal-reached notification with the user PC device, if the current biometric measurement matches the desired biometric characteristic.

17. The method of monitoring glucose levels for weight loss, the method as claimed in claim 1 comprises the steps of:
providing a social network managed by the remote server, wherein the user account is associated to a user profile on the social network;
providing at least one meal picture in the user biometric profile; and
posting the meal picture and the goal-reached notification on the user profile with the remote server.

18. The method of monitoring glucose levels for weight loss, the method as claimed in claim 17 comprises the steps of:
providing a plurality of other accounts managed by the remote server, wherein the plurality of other accounts is associated to an advanced biometric history;
comparing the user biometric profile to the advanced biometric history of each of the plurality of other accounts in order to identify at least one biometrically-similar account from the plurality of other accounts; and
outputting a projective portion for the advanced biometric history of the biometrically-similar account with the user PC device.
